(19) United States
(12) Patent Application Publication
(10) Pub. No.: US 2009/0131760 A1
(43) Pub. Date: May 21, 2009

(54) Morphograms In Different Time Scales For Robust Trend Analysis In Intensive/Critical Care Unit Patients

(75) Inventors: Walid S. I. Ali, Chandler, AZ (US); Mohammed Saeed, Cambridge, MA (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 11/916,771

(22) PCT Filed: May 31, 2006
(86) PCT No.: PCT/IB2006/051744

§ 371 Date: Aug. 26, 2008

Related U.S. Application Data
(60) Provisional application No. 60/688,887, filed on Jun. 9, 2005.

(51) Int. Cl. A61B 5/00

(52) U.S. Cl. 600/301

(57) ABSTRACT

A patient monitoring system that simultaneously analyzes physiological signals from at least one patient monitoring device (4) to detect unstable conditions includes a frequency component extractor (6) that separates each received signal into a plurality of frequency components over different time scales; a generator (8) that provides mappings of physiological signals against one another called morphograms, which show how the physiological signals more together and a processing component (10) that analyzes the morphograms to determine whether an unstable condition exists.

The following relates to patient monitoring, diagnosing, medical alert systems, and the like. It finds particular application to simultaneously analyzing physiological information presented at different time scales to characterize short to long term physiological trends.

Intensive/critical care units (ICU/CCU) patients typically are connected to a plurality patient monitoring devices that continuously or periodically monitor a variety of physiological conditions through patient vital signs such as heart rate and blood pressure, for example. From the monitored vital signs, the patient monitoring devices detect clinically significant events and sound alarms to warn the clinical staff about the clinically significant events with an occasional false alarm.

The vital sign signals within monitored physiological information have a variety of clinically significant periodicities or frequency components. For example, a current pulse rate and an average pulse rate over several time periods (e.g., seconds, minutes, hours, days . . . ) provide short term and long term clinically significant information about the heart and associated organs. Similarly, blood pressure can fluctuate over minutes, hours or days. The number of such frequency components that are clinically significant depends on the particular organ. Because a heart attack occurs in seconds, it is usually detected by monitors that sample on very short intervals. In contrast, a condition such as leakage into the cardiac sac is a condition that manifests over several days and is best diagnosed using a diagnostic tool that monitors trends over many days.

Alarms due to artifacts in such monitored signals reduce efficacy of healthcare provision, especially in intensive and critical care units. Thus, more analysis is needed to increase the fidelity (e.g., quality-truthfulness) of these alarms. Techniques exist that identify events in ECG signals. However, many of these techniques only correct for errors in individual ECG leads of a multi-lead ECG monitor using computationally intensive algorithms. Other techniques fuse more than one source of information to produce a reliable instantaneous understanding of a signal's quality. These methods, however, are complicated and computational intense and are not currently practical for deployment in an ICU/CCU.

In one embodiment, a patient monitoring system that simultaneously analyzes physiological signals from at least one patient monitoring device to detect unstable conditions is illustrated. At least one patient monitoring device monitors physiological information sensed from one or more sensors on a patient. A frequency component extractor separates each received signal into a plurality of frequency components over different time scales. A generator generates interrelationships for each frequency component to create a set of morphograms interrelationships that characterizes short to long term signal relationships. A processing component analyzes the interrelationships to determine whether an unstable condition exists.

One advantage includes simultaneously displaying and analyzing morphograms over different time scales.

Another advantage resides in detecting unstable conditions by locating inconsistencies between morphograms.

Another advantage lies in separating physiological signals into a plurality of frequency components over different time scales.

Another advantage resides in automatically informing clinical staff when an unstable condition is detected.

Another advantage resides in recognition of changes in physiological conditions and trends occurring over a variety of time periods.

Still further advantages will become apparent to those of ordinary skill in the art upon reading and understanding the detailed description of the preferred embodiments.

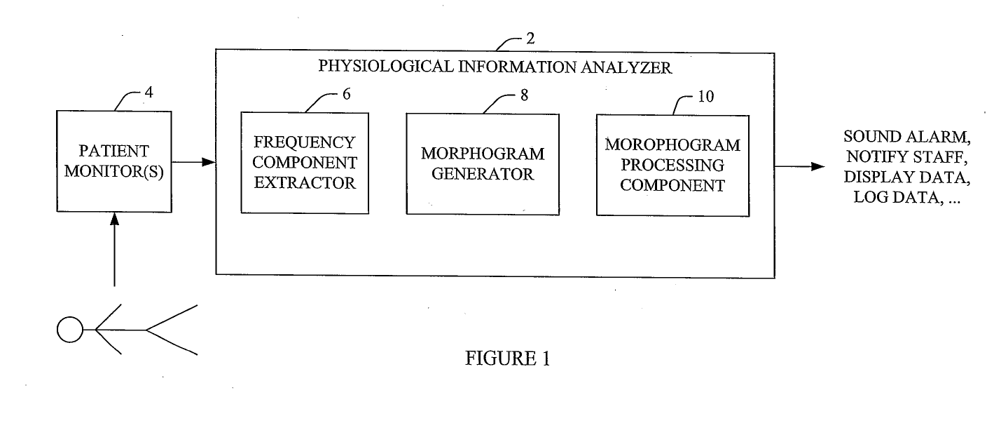

FIG. 1 illustrates a patient monitoring system for analyzing physiological information over a plurality of time.

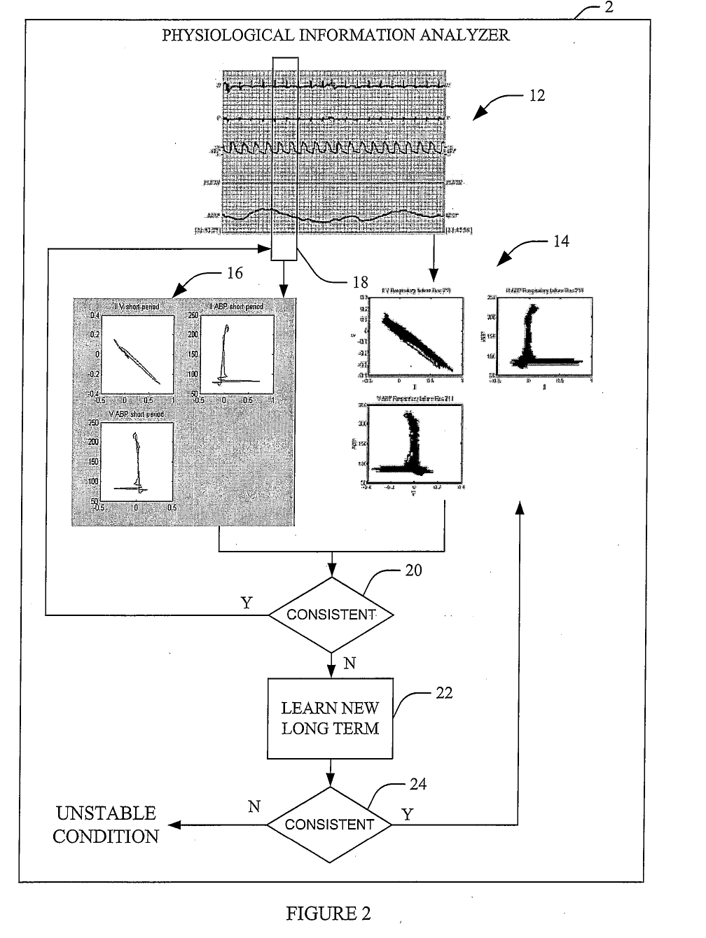

FIG. 2 provides a particular example in which short-term morphograms are compared with long term morphograms to detect an unstable condition in a patient.

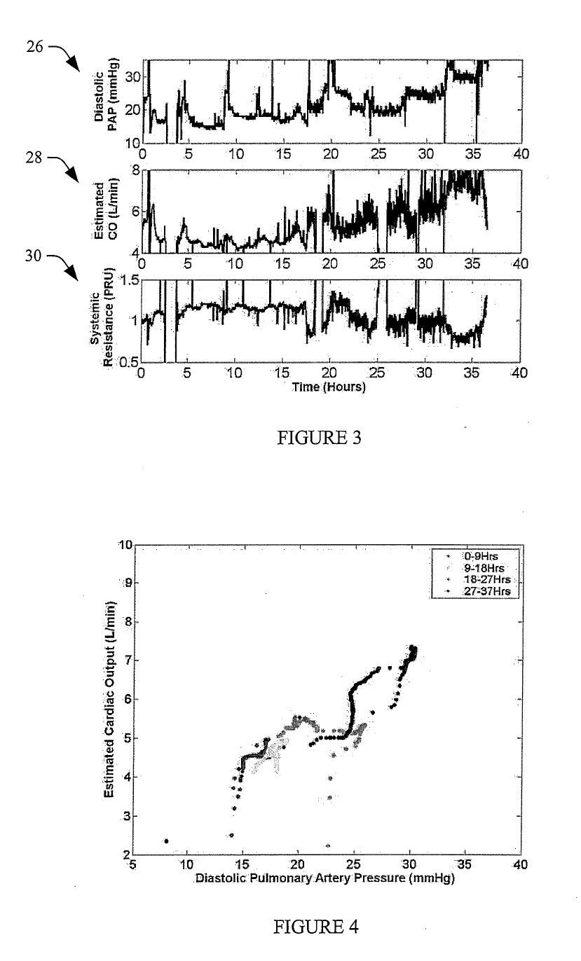

FIGS. 3-6 illustrate various sets of signals over time and corresponding multi-resolution morphograms.

FIG. 1 illustrates a patient monitoring system (“system”) that analyzes physiological information over a plurality of time scales to determine whether a patient's condition is improving, stablizing, or degradating. The system includes a physiological information analyzer 2 that receives physiological information from one or more patient monitors 4 that monitor physiological information obtained from one or more sensors on a patient. For example, ECG leads (sensors) are placed at various positions on the patient to sense electrical activity of the heart, blood pressure monitors sense various pressures such as diastolic pulmonary artery pressure, blood oxygen sensors sense blood oxygen levels, etc.. The sensed signals are collected and processed by one of the patient monitors 4 (e.g., an ECG monitoring device) and used to generate a visual (e.g., graph, value . . . ) and/or audio (e.g., heart rate . . . ) representation indicative of the heart. The patient monitor 4 provides the raw and/or processed signals to the physiological information analyzer 2.

The physiological information analyzer 2 includes a frequency component extractor 6 that receives the physiological information from the patient monitors 4. The frequency component extractor 6 splits each received physiological signal into a plurality of frequency components with different time scales. For example, a signal obtained from a primary ECG lead can be delineated across time periods of fractions of a second, seconds, minutes, hours, days, week, etc. It is to be appreciated that essentially an infinite number of physiological signals representing different physiological information can be received and split into one or more frequency components. Typically, the number of signals received is dependent upon the patient monitoring devices 4, the patient's diagnosis, the attending clinician, and the memory and computation power of the system.

The frequency component extractor 6 employs various techniques to extract the frequency components from a physiological signal. For example, the frequency component extractor 6 can use a Gabor filter, a Fourier transform, a moving average, and the like to extract frequency components. In a preferred embodiment, wavelet decomposition is used to extract the frequency components of the signals. Wavelet transforms resolve a signal into several time scales, and localize the frequency components for analysis of slow and fast moving events.

The physiological information analyzer 2 further includes a morphogram generator 8 that receives the frequency components of the physiological signals and generates one or more morphograms. Morphograms are geometric relationships that easily and efficiently capture the interaction (correlation) of patterns between signals to provide a shape fingerprint for the interaction that describe physiology. More particularly, each morphogram provides a mapping of physiological signals against one another to show how the physiological signals move together. The mapping includes generating coefficients (e.g., horizontal, vertical and diagonal details) that uniquely describe shape variability. The ability of one type of physiological signal to follow another type depends on the correlation between the types of physiological signals. In the patient monitoring domain, the morphogram depicts the relationship between physiological data such as an ECG signal and an arterial blood pressure (ABP) signal.

This mapping can be achieved using tools such as plot or graphs. For instance, ECG data (e.g., coefficients resulting from the wavelet decomposition) can be mapped to one axis and ABP data (e.g., coefficients resulting from the wavelet decomposition) can be mapped to a remaining axis, where the relationship is visually depicted through a graph. It is to be understood that three, four, . . . , N dimensional graphs, where N is an integer equal to or greater than one, can be generated. Morphograms representing different time scales can be individually plotted on different graphs or superimposed onto a single graph. In another instance, the physiological data is used to generate an equation that characterizes the relationship.

The physiological information analyzer 2 further includes a morphogram processing component 10 that processes the morphograms and constructs trends for respective time scales. The morphogram processing component 10 compares trends of data acquired from different patient monitoring devices 4 within a similar time scale (e.g., all data at X second intervals, where X is a real positive number) and data acquired from similar and different patient monitoring devices 4 across time scales (e.g. data from X, M, L, etc. time periods, where X, M, and L are positive real numbers and are not equal). By way of example, the morphogram processing component 10 simultaneously compares ECG-ECG morphograms at time scales for K seconds, minutes, hours, etc., where K is a positive real number.

Stable morphograms typically correspond to a stable physiological state. Degenerating or changing morphograms typically connote a degenerating or changing physiological state. The morphograms comparisons facilitate determining patterns (regions of stability) and detecting differences and/or changes, and are used to determine whether the patient's condition is improving, stablizing, or degradating. For example, a heart arrhythmia can be detected by comparing snapshots (morphogram) of the heart's electrical behavior over similar time scales that were recorded at different times (e.g., yesterday vs today). Changes in such morphograms can indicate a change in the heart's condition, for example, due to myocardial infarction. Consistency between the morphograms represents stability. Likewise, snapshots at different time scales (e.g., long time scale and short time scale) can indicate deterioration or improvement. Long time scale morphograms typically represent a good approximation of the structure of the signals. They emphasize steady-states, level changes and trends occurring on the data. Short time scale morphograms represent current behavior. Thus, a short time scale morphogram that is inconsistent with a long time scale morphogram (or steady-state) can indicate deterioration or a change in steady-state.

Various techniques can be used to measure stability, consistency, etc. between morphograms. Suitable techniques to describe similarities between morphogram signatures include approximating the centroids of the morphograms in one or several dimensions, two-dimensional template matching, wavelet decompositions, area bounds, and two dimensional fourrier and discrete cosine transforms, for example. In one embodiment, an absolute difference between the centroids of two or more morphograms are compared to a threshold, C_diff, and if the threshold is exceeded, then the morphograms are not deemed to be consistent with one another.

The results of the analysis are used to invoke various responses. For example, in one instance the morphograms can be visually displayed for visual inspection by clinical staff. Such visualization can present the morphograms concurrently or serially. For instance, multiple graphs can be displayed for simultaneous observation of morphograms at different time scales. In another instance, multiple plots can be superimposed on the same graph for simultaneous observation of morphograms at different time scales. In yet another instance, individual morphograms can be scrolled through. In another example, the change in physiological condition as determined by the analysis of the different time scale morphograms can elicit an alarm (e.g., a patient monitor alarm, a bed-side or remote monitoring station alarm . . . ). In yet another example, healthcare staff can automatically be paged (e.g., beeper, cell phone, office phone, email, over an intercom system . . . ) in response a change deemed significant. In another example, the data can be simply stored or logged for retroactive analysis. In another example, the morphograms and trends are compared with characteristic morphograms and trends in a diagnosis memory to retrieved a corresponding diagnosis.

FIG. 2 provides a particular example in which short-term (time scale) morphograms are compared with long term (time scale) morphograms to detect an unstable condition in a patient. A plurality of hemodynamic and ECG signals (illustrated at 12) are received by the physiological information analyzer 2. In this example, the entire time length of the signals is used to construct long time scale morphograms 14. As noted previously, long time scale morphograms represent approximations of the signals and emphasize steady-states, level changes and trends occurring on the data. Short time scale morphograms 16 are generated from data within a time slice window 18. The width and step size of the window can be variously configured to achieve a desired resolution.

Upon generating a set of short time scale morphograms, the short time scale morphograms are compared at 20 with the long time scale morphograms. If the short time scale morphograms are consistent with the long time scale morphograms, the time slice window 18 moves based on its step size and a new set of short time scale morphograms is generated. If the short time scale morphograms are inconsistent with the long time scale morphograms, a new set of long time scale morphograms are generated at 22. The short time scale morphograms are then compared at 24 with the new long time scale morphograms. If the short and new long time scale morphograms are consistent, the new long time scale morphograms replace the existing long time scale morphograms 14. This new set of long time scale morphograms represents the new steady state. If the short and new long time scale morphograms are inconsistent, the physiological information analyzer 2 determines an unstable condition may exist. Health care staff can be notified as described above. Optionally, the information can be further processed to determined whether the unstable condition is due to a clinically significant physiological change or an artifact.

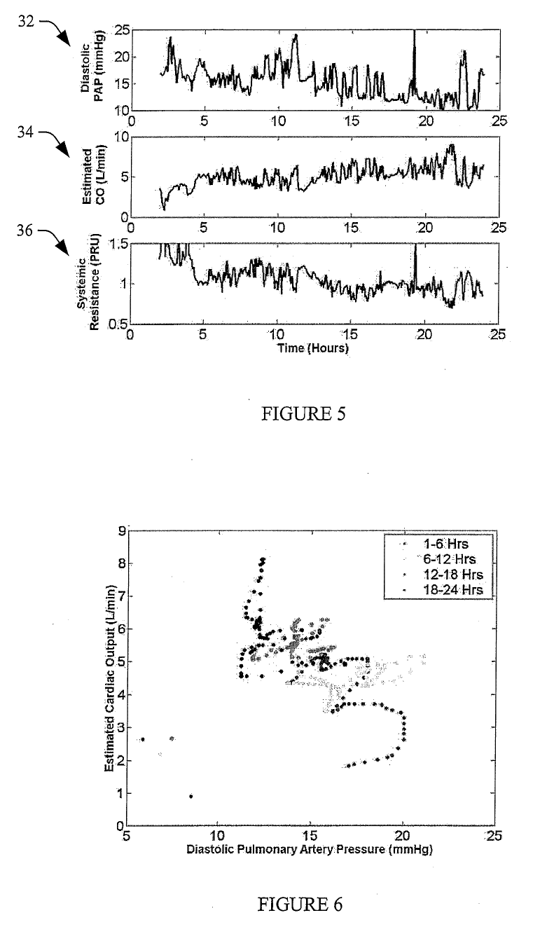

FIGS. 3-6 illustrate various sets of signals over time and corresponding multi-resolution morphograms. FIG. 3 shows a diastolic signal 26, an estimated cardiac output (CO) signal 28 and a systemic resistance signal 30 over time. FIG. 4 simultaneously show corresponding estimated cardiac output - diastolic pulmonary artery pressure morphograms for four different time scales: 0-9 hours, 9-18 hours, 18-27 hours, and 27-37 hours. FIG. 5 shows another set of signals, a diastolic signal 32, an estimated CO signal 34 and a systemic resistance signal 36 over time. FIG. 6 show corresponding estimated cardiac output - diastolic pulmonary artery pressure morphograms over time scale of 0-6 hours, 6-12 hours, 12-18 hours, and 18-24 hours.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A patient monitoring system that simultaneously analyzes physiological signals from at least one patient monitoring device that monitors physiological information sensed from one or more sensors on a patient to detect unstable conditions, comprising:
a frequency component extractor (that separates each received signal into a plurality of frequency components over different time scales;
a generator that generates an interrelationship for each frequency component to create a set of interrelationships that characterizes short to long term signal relationships; and
a processing component that analyzes the interrelationships to determine whether an unstable condition exists.

2. The patient physiological information monitoring system as set forth in claim 1, the interrelationships are morphograms.

3. The patient physiological information monitoring system as set forth in claim 2, further including:
a display that simultaneously presents morphograms for user visualization in one of a multi-resolution format in which multiple morphograms are superimposed in a single graph or multiple morphograms are individually displayed in separate graphs.

4. The patient physiological information monitoring system as set forth in claim 2, further including a component that determines whether an unstable condition is due to a clinically significant ever or an artifact.

5. The patient physiological information monitoring system as set forth in claim 2, wherein the morphogram processing component in response to detecting an unstable condition performs at least one of the following: notifies clinical staff; invokes an alarm; logs results; and displays results.

6. The patient physiological information monitoring system as set forth in claim 2, wherein the frequency component extractor utilizes one or more of the following to extract the frequency components: a Fourier transform, a Gabor filter, a moving average, and a wavelet transform.

7. The patient physiological information monitoring system as set forth in claim 2, wherein the frequency component extractor uses a wavelet transform to localize the frequency components for simultaneous analysis of slow and fast moving events.

8. The patient physiological information monitoring system as set forth in claim 2, the morphograms are represented through equations.

9. The patient physiological information monitoring system as set forth in claim 1, the interrelationships are depicted as characteristics relating two signals through horizontal, vertical and diagonal coefficients.

10. The patient physiological information monitoring system as set forth in claim 1, the morphogramn processing component generates morphograms that emphasize at least one of steady-states, level changes, and trends.

11. A method for detecting physiological unstable conditions, comprising:
receiving physiological information from the at least one patient monitoring device;
separating the physiological information by frequency components that represent different time scales;
generating a interrelationships for every pair of signals at each time scale; and
simultaneously analyzing the interrelationships across time scales to detect unstable conditions.

12. The method as set forth in claim 11, the interrelationships are morphograms.

13. The method as set forth in claim 12, further including:
comparing a set of short time-scale morphograms with a set of long time-scale morphograms that represent steady state; and
determining stability based on consistency of the short time-scale morphograms with the long time-scale morphograms.

14. The method as set forth in claim 13, further including:
generating a new set of long time-scale morphograms;
comparing the set of short time-scale morphograms with the new set of long time-scale morphograms;
determining stability based on consistency of the short time-scale morphograms with the new long time-scale morphograms; and
replacing the set of long time-scale morphograms with the new set of long time-scale morphograms to represent the steady state.

15. The method as set forth in claim 12, further including:
comparing a set of short time-scale morphograms with a set of long time-scale morphograms that represent steady state;
determining potential instability when the set of short timescale morphograms are inconsistent with the set of long time-scale morphograms;
generating a new set of long time-scale morphograms;
comparing the set of short time-scale morphograms with the new set of long time-scale morphograms;
determining instability when the set of short time-scale morphograms are inconsistent with the new set of long time-scale morphograms; and
notifying clinical staff of the unstable condition.

16. The method as set forth in claim 15, further including simultaneously analysing the morphograms of different time-scales to determine whether the instability is due to a clinically significant physiological change or an artifact.

17. The method as set forth in claim 12, further including simultaneously displaying morphograms in different time for visualization by clinical staff.

18. The method as set forth in claim 11, further including at least one of invoking an alarm; logging results; and notifying clinical staff when an unstable condition is detected.

19. The method as set forth in claim 11, further including using wavelet decomposition to extract the frequency components from the physiological information.

20. A computer programmed to perform the method of claim 11.

21. A method of patient monitoring, comprising:
generating a plurality of signals indicative of an evolving physiological state of a subject;
decomposing the signals into a plurality of frequency-based time scales; and analyzing relationships between the signals of different time-scales to detect or predict changes in the evolving physiological state.
